(19) United States
(12) Patent Application Publication
(10) Pub. No.: US 2007/0275100 A1
(43) Pub. Date: Nov. 29, 2007

(54) Use of gaseous nitric oxide as an anti-cancer agent

(76) Inventor: Christopher C. Miller, North Vancouver (CA)

Correspondence Address: SIDLEY AUSTIN BROWN & WOOD LLP (LAIP GROUP), 555 W. FIFTH ST., SUITE 4000, LOS ANGELES, CA, 90013, US

(21) Appl. No.: 11/704,791

(22) Filed: Feb. 9, 2007

Related U.S. Application Data
(63) Continuation-in-part of application No. 11/211,055, filed on Aug. 23, 2005.
Application No. 11/211,055 is a continuation of application No. 09/762,152, filed on Feb. 1, 2001, now abandoned.

(30) Foreign Application Priority Data
Nov. 23, 1998 (CA) 2,254,645

(51) Int. Cl. A61K 33/00

(52) U.S. Cl. 424/718

(57) ABSTRACT

The invention relates to a method for treating, controlling, or preventing cancerous cell phenotypes and growths in an animal involving the administration of gaseous nitric oxide to one or administration sites in a body. The invention generally is capable of treating cancers found in or on the adrenal gland, bladder, bones, brain, breast, cervix, colon, colorectum, esophagus, gastrointestinal tract, heart, kidney, liver, large intestine, lungs, mouth, ovaries, pancreas, parathyroid, pituitary gland, prostate, salivary gland, skin, small intestine, spleen, stomach, thymus, thyroid, testicles, urinary tract, uterus, vagina, and so forth.

### CLAIM OF PRIORITY

This application claims priority to U.S. patent application Ser. No. 11/211,055, filed on Aug. 23, 2005, pending before the U.S. Patent and Trademark Office, which claims priority to U.S. patent application Ser. No. 09/762,152, filed Feb. 1, 2001, now abandoned. Ser. No. 09/762,152 claims priority to Canadian Patent Application No. 2,254,645, filed on Nov. 23, 1998, expressly incorporated by reference in their entirety. Ser. No. 11/211,055 and Ser. No. 09/762,152 also are expressly incorporated herein by reference in their entirety.

### FIELD OF THE INVENTION

The invention generally relates to the use of gaseous nitric oxide (gNO) to treat various ailments in animal bodies. More particularly, the invention relates to the use of gNO to treat cancerous and pathogenic cells in animal, and preferably mammalian, and more preferably human, bodies. Additionally, the invention relates to devices and methods for delivering gNO to administration sites in the body.

### BACKGROUND OF THE INVENTION

In healthy humans, endogenously synthesized nitric oxide (NO) is thought to exert an important mycobacteriocidal or inhibitory action in addition to a vasodilatory action. There have been a number of ongoing, controlled studies to ascertain the benefits, safety and efficacy of inhaled nitric oxide as a pulmonary vasodilator. Inhaled nitric oxide has been successfully utilized in the treatment of various pulmonary diseases such as persistent pulmonary hypertension in newborns and adult respiratory distress syndrome. There has been no attempt, however, to reproduce the mycobacteriocidal or inhibitory action of NO with exogenous NO.

Gaseous NO itself has proven to be difficult to administer in some applications as it is a highly reactive gas and may cause hypotension if administered systemically.

Further background information relating to the present invention may be found in the following references:
Lowenstein, C. J., J. L. Dinerman, and S. H. Snyder. 1994. Nitric oxide: a physiologic messenger” Ann. Intern. Med. 120:227-237.
The neonatal inhaled nitric oxide study group. 1997. Inhaled nitric oxide in full-term and nearly full-term infants with hypoxic respiratory failure. N. Engl. J. Med. 336:597-604.
Roberts, J. D. Jr., J. R Fineman, F. C. Morin III, et al. for the inhaled nitric oxide study group. 1997. Inhaled nitric oxide and persistent pulmonary hypertension of the newborn. N. Engl. J. Med. 336:605-6 10.
Rossaint, R., K. J. Falke, F. Lopez, K. Slama, U. Pison, and W. M. Zapol. 1993. Inhaled nitric oxide for the adult respiratory distress syndrome. N. Engl. J. Med. 328:399-405.
Rook, G. A. W. 1997. Intractable mycobacterial infections associated with genetic defects in the receptor for interferon gamma: what does this tell us about immunity to mycobacteria? Thorax. 52 (Suppl 3):S41-S46.
Denis, M. 1991. Interferon-gamma-treated murine macrophages inhibit growth of tubercle bacilli via the generation of reactive nitrogen intermediates. Cell. Immunol. 132:150-157.
Chan, J., R. Xing, R. S. Magliozzo, and B. R. Bloom. 1992. Killing of virulent Mycobacterium tuberculosis by reactive nitrogen intermediates produced by activated murine macrophages. J. Exp. Med. 175:1111-1122.
Chan, J., K. Tanaka, D. Carroll, J. Flynn, and B. R. Bloom. 1995. Effects of nitric oxide synthase inhibitors on murine infection with Mycobacterium tuberculosis. Infect. Immun. 63:736-740.
Nozaki, Y., Y. Hasegawa, S. Ichiyama, I. Nakashima, and K. Shimokata. 1997. Mechanism of nitric oxide-dependent killing of Mycobacterium bovis BCG in human alveolar macrophages. Infect. Immun. 65:3644-3 647.
Canetti, G. 1965. Present aspects of bacterial resistance in tuberculosis. Am. Rev. Respir. Dis. 92:687-703.
Hendrickson, D. A., and M. M. Krenz. 1991. Regents and stains, P. 1289-1314. In Balows, A, W. J. Hausler Jr., K. L. Herrmann, H. D. Isenberg, and I-Ii. Shadomy (eds.), Manual of Clinical Microbiology, 5th ed., 1991. American Society for Microbiology, Washington, D.C.
Szabo, C. 1996. The pathophysiological role of peroxynitrite in shock, inflammation and ischemia-reperfusion injury. Shock. 6:79-88.
Stavert, D. M., and B. E. Lehnert. 1990. Nitrogen oxide and nitrogen dioxide as inducers of acute pulmonary injury when inhaled at relatively high concentrations for brief periods. Inhal. Toxicol. 2:53-67.
Hugod, C. 1979. Effect of exposure to 43 ppm nitric oxide and 3.6 ppm nitrogen dioxide on rabbit lung. mt. Arch. Occup. Environ. Health. 42:159-167
Frostell, C., M. D. Fratacci, J. C. Wain, R. Jones and W. M. Zapol. 1991. Inhaled nitric oxide, a selective pulmonary vasodilator reversing hypoxic pulmonary vasoconstriction. Circulation. 83:2038-2047.
Bult, H., G. R. Y. Dc Meyer, F. H. Jordaens, and A. G. Herman. 1991. Chronic exposure to exogenous nitric oxide may suppress its endogenous release and efficacy. J. Cardiovasc. Pharmacol. 17: S79-S82.
Buga, G. M., J. M. Griscavage, N. E. Rogers, and L. J. Ignarro. 1993. Negative feedback regulation of endothelial cell function by nitric oxide. Circ. Res. 73:808-8 12
Assreuy, J., F. Q. Cunha, F. Y. Liew, and S. Moncada. 1993. Feedback inhibition of nitric oxide synthase activity by nitric oxide. Br. J. Pharmacol. 108:833-837.
O'Brien, L., J. Carmichael, D. B. Lowrie and P. W. Andrew. 1994. Strains of Mycobacterium tuberculosis differ in susceptibility to reactive nitrogen intermediates in vitro. Infect. Immun. 62:5187-5190.
Long, R., B. Maycher, A. Dhar, J. Manfreda, E. Hershfield, and N. R. Anthonisen. 1998. Pulmonary tuberculosis treated with directly observed therapy: serial changes in lung structure and function. Chest. 113:933-943.
Bass, H., J. A. M. Henderson, T. Heckscher, A. Oriol, and N. R. Anthonisen. 1968. Regional structure and function in bronchiectasis., Am. Rev. Respir. Dis. 97:598-609.
De Groote M A, Fang F C. 1995. NO inhibitions: antimicrobial properties of nitric oxide. Clinical Infectious Diseases; 21 (suppl 2): S162-165.

The description herein of problems and disadvantages of known apparatus, methods, and devices is not intended to limit the invention to the exclusion of these known entities. Indeed, embodiments of the invention may include one or more of the known apparatus, methods, and devices without suffering from the disadvantages and problems noted herein.

### SUMMARY OF THE INVENTION

There is a need for a more effective method to treat pathogenic conditions in animal bodies. Additionally, there is a need for a more effective procedure to administer gaseous NO to treat various ailments in animal bodies. Furthermore, there is a need for a more targeted method of treating cancerous cell phenotypes in animal bodies. Also, there is a need for devices to more effectively deliver gaseous NO in treatment regimes, and especially for a method of delivering gaseous NO to target sites without damaging healthy collateral host cells and while simultaneously identifying the target site and evaluating the effect of the gaseous NO administration thereto.

Accordingly, there is provided a method for preventing cancerous cell phenotypes and growths in an animal. The method comprises providing gaseous nitric oxide and administering the gaseous nitric oxide to the animal at one or more administration sites. There also is provided a method for eradicating cancerous cell phenotypes and growths in an animal comprising providing gaseous nitric oxide and administering the gaseous nitric oxide to the animal at one or more administration sites. Both methods optionally include scavenging gaseous nitric oxide from the one or more administration sites. The method for preventing cancerous cell phenotypes and growths in an animal and the method of eradicating cancerous cell phenotypes and growths in an animal may be carried out using gaseous nitric oxide administered in a concentration from about 200 ppm and higher.

Another embodiment is a method for eradicating cancerous cell phenotypes and growths in an animal comprising the steps of: (1) identifying cancerous cell phenotypes or growths in the mammal with a device; and (2) administering the gaseous nitric oxide to the identified cancerous cell phenotypes or growth with the device, wherein the gaseous nitric oxide is administered at a concentration greater than or equal to about 200 ppm.

These and other features and advantages of the embodiments will be apparent from the description provide herein.

### BRIEF DESCRIPTION OF THE DRAWINGS

The nature and scope of the invention will be elaborated in the detailed description which follows, in connection with the enclosed drawing figures, in which:

FIG. 1, embodiment A, shows the external features of a pulse-dose delivery device for nitric oxide according to the present invention;

FIG. 1, embodiment B, illustrates schematically the internal working components of the device of FIG. 3a;

FIG. 2 is a schematic illustration of the specialized valve used to control the delivery of nitric oxide in a preset dosage through the disposable nasal cannula of a device according to the present invention;

FIG. 3 is a schematic drawing of the mask-valve arrangement of a pulsed-dose nitric oxide delivery device according to the present invention;

FIG. 4 illustrates a plate exposed to Staphylococcus aureus and 25,000 ppm gNO for 1 minute prior to extended incubation;

FIG. 5 illustrates a plate exposed to Staphylococcus aureus and 25,000 ppm gNO for 3 minutes prior to extended incubation;

FIG. 6 illustrates a control plate exposed to Staphylococcus aureus and air prior to extended incubation;

FIG. 7 illustrates the cellular sensitivity of A549, NCI-H23, NCI-H460, HTB-58, H2170, and H441 cell lines to 25,000 ppm of gNO for 1 minute, 3 minutes, 6 minutes, 9 minutes, 10 minutes, 20 minutes, and 30 minutes;

FIG. 8, embodiments A-D, illustrates the effect of administering 25,000 ppm gNO to, respectively, normal human bronchial epithelial cells (NHBE), normal human endothelial lung vascular cells (NHVE), normal human fibroblast cells (NHLF), and squamous lung cancer cells (H2170);

FIG. 9, embodiments A and B, illustrates an exemplary delivery device for the local administration of gNO;

FIG. 10, embodiments A and B, illustrates another exemplary delivery device for the local administration of gNO; and

FIG. 11 illustrates an exemplary pumping mechanism for delivering gNO to a delivery device for the nearly-continuous local administration of gNO.

### DETAILED DESCRIPTION OF THE PREFERRED EMBODIMENTS

As used throughout this disclosure, the singular forms “a,” “an,” and “the” include plural reference unless the context clearly dictates otherwise.

All technical and scientific terms used herein have the same meanings as commonly understood by one of ordinary skill in the art to which this invention belongs. All publications mentioned herein are cited for the purpose of describing and disclosing the embodiments. Nothing herein is to be construed as an admission that the embodiments described are not entitled to antedate such disclosures by virtue of prior invention.

Before the present compositions and methods are described, it is to be understood that this invention is not limited to the particular devices, compositions, methodologies or protocols described, as these may vary. It is also to be understood that the terminology used in the description is for the purpose of describing the particular versions or embodiments only, and is not intended to limit the scope of the present invention which will be limited only by the appended claims. For simplicity, each reference referred to herein shall be deemed expressly incorporated by reference in its entirety as if fully set forth herein.

Methods and devices disclosed herein may be useful for preventing cancerous cell phenotypes and growths in animal, and preferably mammal, and more preferably human, bodies. As used herein, “preventing” includes inhibiting the growth, spread, and development of cancerous cell phenotypes and growths. Methods and devices disclosed herein also may be useful for eradicating cancerous cell phenotypes and growths in animal, and preferably mammal, and more preferably human, bodies. As used herein, “eradicating” includes treating, controlling, suppressing, hindering, blocking, killing, and slowing the spread or development of cancerous cell phenotypes and growths.

The invention also relates to delivery devices for the local administration of gNO to administrations sites in an animal, preferably a mammal, and more preferably a human, body. The delivery devices may provide gNO to the administration sites in a manner that reduces the harm done to adjacent healthy host cells. The invention further relates to a pumping mechanism that may be used to provide a nearly continuous flow of gNO to delivery devices such as those described herein.

Additionally, the invention relates to methods of delivering high-dosage gNO to mammals, and more preferably to humans. The high-dosage gNO delivery methods may be useful, for example, in preventing or eradicating cancerous cell phenotypes and growths.

In another aspect of the invention, the invention relates to a method for suppressing pathogenic cells, and a method for treating an animal having pathogenic cells in its respiratory tract, utilizing a source of nitric oxide. More particularly, the invention relates to a method for suppressing pathogenic cells comprising the step of exposing the pathogenic cells to an effective amount of a nitric oxide source. Further, the invention relates to a method for treating an animal having pathogenic cells in the respiratory tract of the animal comprising the step of delivering by the inhalation route to the respiratory tract of the animal an effective amount of a nitric oxide source.

In another aspect of the invention, the invention relates to a use and a therapeutic use of a source of nitric oxide for suppressing or treating pathogenic cells. More particularly, the invention relates to the use of an effective amount of a nitric oxide source for suppressing pathogenic cells exposed thereto. Further, the invention relates to the therapeutic use of an effective amount of a nitric oxide source for the treatment by the inhalation route of an animal having pathogenic cells in the respiratory tract of the animal. Preferably, as discussed further below, the present invention relates to the novel use of inhaled nitric oxide gas as an agent for killing bacterial cells, parasites and fungi in the treatment of respiratory infections.

In another of the invention, the invention relates to a pharmaceutical composition for use in treating an animal having pathogenic cells in its respiratory tract, which composition comprises a nitric oxide source. More particularly, in this aspect of the invention, the invention relates to a pharmaceutical composition for use in the treatment by the inhalation route of an animal having pathogenic cells in the respiratory tract of the animal, the pharmaceutical composition comprising an effective amount of a nitric oxide source.

In another aspect of the invention, the invention relates to an apparatus or device for supplying, delivering or otherwise providing a nitric oxide source. Preferably, the apparatus or device provides the nitric oxide source for the particular applications, methods and uses described herein. However, the apparatus or device may also be used for any application, method or use requiring the supply, delivery or provision of a nitric oxide source.

In some aspects of the invention, the nitric oxide source is preferably nitric oxide per se, and more particularly, nitric oxide gas. However, alternately, the nitric oxide source may be any nitric oxide producing compound, composition or substance. In other words, the nitric oxide source may be any compound, composition or substance capable of producing or providing nitric oxide, and particularly, nitric oxide gas. For instance, the compound, composition or substance may undergo a thermal, chemical, ultrasonic, electrochemical or other reaction, or a combination of such reactions, to produce or provide nitric oxide to which the pathogenic cells are exposed. As well, the compound, composition or substance may be metabolized within the animal being treated to produce or provide nitric oxide within the respiratory tract of the animal.

Further, in all aspects of the invention, the invention is for use in suppressing or treating any pathogenic cells. For instance, the pathogenic cells may be tumor or cancer cells. However, in one aspect of the invention the pathogenic cells are preferably pathogenic microorganisms, including but not limited to pathogenic bacteria, pathogenic parasites and pathogenic fungi. More preferably, the pathogenic microorganisms are pathogenic mycobacteria. In a preferred embodiment, the pathogenic mycobacteria is M. tuberculosis.

Referring to the use of the nitric oxide source and method for suppressing pathogenic cells using the nitric oxide source, as indicated, the nitric oxide source is preferably nitric oxide per se. However, the nitric oxide source may be a compound, composition or substance producing nitric oxide. In either event, the pathogenic cells are suppressed by the nitric oxide. Suppression of the pathogenic cells by nitric oxide may result in either or both of an inhibitory effect on the cells and a cidal effect on the cells. However, preferably, the nitric oxide has a cidal effect on the pathogenic cells exposed thereto. Thus, it has been found that these aspects of the invention have particular application for the sterilization of medical and other equipment, instruments and devices requiring sterilization.

As well, the pathogenic cells may be exposed to the nitric oxide and the exposing step of the method may be performed in any manner and by any mechanism, device or process for exposing the pathogenic cells to the nitric oxide source, and thus nitric oxide, either directly or indirectly. However, in the preferred embodiment, the pathogenic cells are directly exposed to the nitric oxide. As a result, where desired, the effect of the nitric oxide may be localized to those pathogenic cells which are directly exposed thereto.

Similarly, the therapeutic use, method for treating and pharmaceutical composition for treatment all deliver the nitric oxide source to the pathogenic cells in the respiratory tract of the animal. The therapeutic use, method and composition may be used or applied for the treatment of any animal, preferably a mammal, including a human. Further, as indicated, the nitric oxide source in these instances is also preferably nitric oxide per se, however, the nitric oxide source may be a compound, composition or substance producing nitric oxide within the respiratory tract. In either event, the nitric oxide similarly suppresses the pathogenic cells in the respiratory tract of the animal. This suppression of the pathogenic cells may result in either or both of an inhibitory effect on the cells and a cidal effect on the cells. However, preferably, the nitric oxide has a cidal effect on the pathogenic cells in the respiratory tract exposed thereto.

As well, the pathogenic cells in the respiratory tract of the animal may be treated by nitric oxide and the delivering step of the therapeutic method may be performed in any manner and by any mechanism, device or process for delivering the nitric oxide source, and thus nitric oxide, either directly or indirectly to the respiratory tract of the animal. In the preferred embodiments of these aspects of the invention, the nitric oxide source is delivered directly by the inhalation route to the respiratory tract of the animal, preferably by either the spontaneous breathing of the animal or by ventilated or assisted breathing.

Further, in some preferred embodiments of these aspects of the invention, the pathogenic cells in the respiratory tract of the animal are treated by, and the delivering step of the therapeutic method is comprised of, exposing the pathogenic cells to the nitric oxide source, and thus nitric oxide, either directly or indirectly. More preferably, the pathogenic cells are directly exposed to the nitric oxide. As a result, where desired, the effect of the nitric oxide may be localized to those pathogenic cells which are directly exposed thereto within the respiratory tract of the animal.

In addition, in all aspects of the invention, an effective amount of the nitric oxide source is defined by the amount of the nitric oxide source required to produce the desired effect of the nitric oxide, either inhibitory or cidal, on the pathogenic cells. Thus, the effective amount of the nitric source will be dependent upon a number of factors including whether the nitric oxide source is nitric oxide per se or a nitric oxide producing compound, the desired effect of the nitric oxide on the pathogenic cells and the manner in which the pathogenic cells are exposed to or contacted with the nitric oxide. In the preferred embodiments of the various aspects of the invention, the effective amount of the nitric oxide source is the amount of nitric oxide required to have a cidal effect on the pathogenic cells exposed directly thereto. Thus, the effective amount for any particular pathogenic cells will depend upon the nature of the pathogenic cells and can be determined by standard clinical techniques. Further, the effective amount will also be dependent upon the concentration of the nitric oxide to which the pathogenic cells are exposed and the time period or duration of the exposure.

Preferably, the pathogenic cells are exposed to a gas or a gas is delivered to the respiratory tract of the animal being treated, wherein the gas is comprised of the nitric oxide source. More preferably, the pathogenic cells are exposed to a gas comprised of nitric oxide. For instance, the gas may be comprised of oxygen and nitric oxide for delivery by the inhalation route to the respiratory tract of the animal being treated.

In another aspect of the invention, the apparatus or device is preferably comprised of a portable battery-operated, self-contained medical device that generates its own nitric oxide source, preferably nitric oxide gas, as a primary supply of nitric oxide. Further, the device may also include a conventional compressed gas supply of the nitric oxide source, preferably nitric oxide gas, as a secondary back-up system or secondary supply of nitric oxide.

Further, the device preferably operates to deliver nitric oxide in the gaseous phase to spontaneously breathing or to ventilated individual patients having microbial infections, by way of a specially designed nasal-cannula or a mask having a modified Fruman valve. In the preferred embodiment, nitric oxide gas is produced in cartridges through thermal-chemical, ultrasonic and/or electrochemical reaction and is released upon user inspiratory demand in pulsed-dose or continuous flow.

Studies of the Applicant on the exposure of extra cellular M. tuberculosis to concentrations of NO for periods have led to the conclusion that exogenous NO exerts a powerful dose-dependent and time-dependent mycobacteriocidal action. Further, it may be inferred that the large population of extracellular bacilli in patients with cavitary pulmonary tuberculosis are also vulnerable to exogenous (inhaled) NO.

Measurements of Cidal Activity of Exogenous gNO

Recent studies have shown an effective dosage of gaseous nitric oxide on bacteria is from about 100 ppm to about 250 ppm, preferably about 200 ppm, such as the data shown in “The Antimicrobial Effect of Nitric Oxide on the Bacteria That Cause Nosocomial Pneumonia in Mechanically Ventilated Patients in the Intensive Care Unit,” B. McMullin, D. R. Chittock, D. L. Roscoe, H. Garcha, L. Wang, and C. C. Miller, Respiratory Care, November 2005, Vol. 50, No. 11, incorporated herein by reference in its entirety.

For the experiment described in the above referenced article, 200 ppm of gNO was applied for 5 hours to Klebsiella pneumoniae, Serratia marcescens, Enterobacter aerogenes, Stenotrophomonas maltophilia, and Acinetobacter baumanii. Additionally, S. aureus (ATCC 25923), P. aeruginosa (ATCC 27853), methicillin-resistant S. aureus, S. aureus, E. coli, and Group B streptococci source colonies were tested from laboratory culture collections.

Continuous in vitro exposure of microorganisms to 200 ppm gNO was cytocidal, within 5 hours, to all the bacteria that cause nosocomial pneumonia in the intensive care unit.

Studies illustrated in “A direct nitric oxide gas delivery system for bacterial and mammalian cell cultures,” A. Ghaffari, D. H. Neil, A. Ardakani, J. Road, A. Ghahary, C. C. Miller. Nitric Oxide 12(3):129-140, 2005, herein incorporated by reference in its entirety, also illustrate the effectiveness of gNO against bacteria.

Primary Unit of the NO Post-Delivery Device

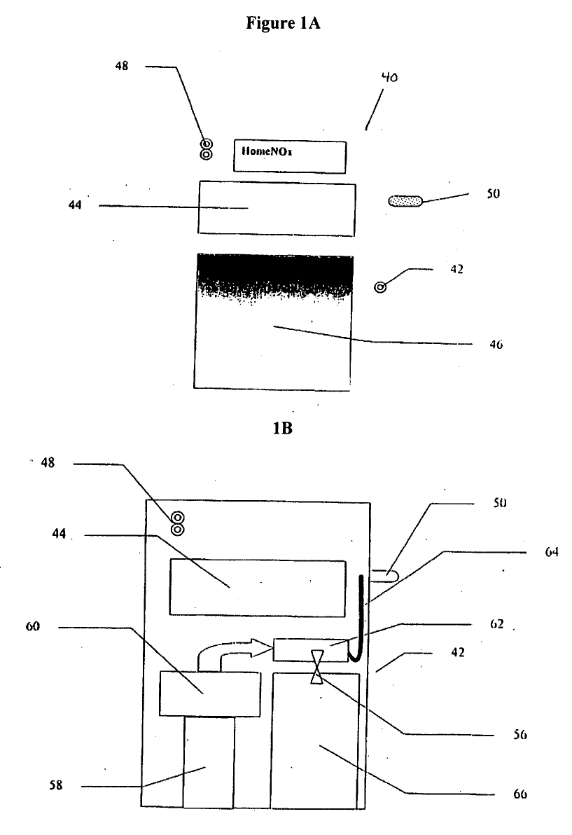

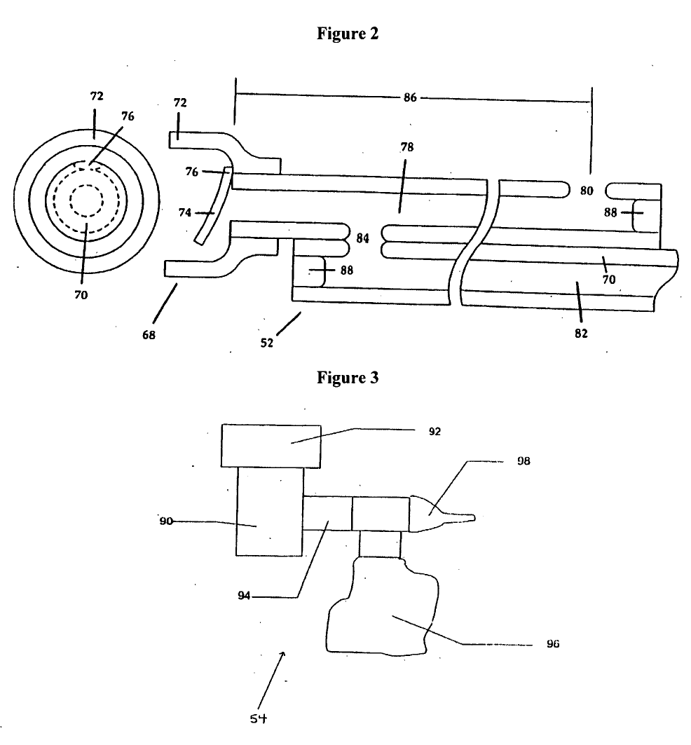

Referring to FIGS. 1a and 1b, the main unit (40) provides a small enclosure designed to hang on a belt. An A/C inlet (42) provides an electrical port to provide power to an internal rechargeable battery which powers the unit (40) if required. The user interface provides a multi-character display screen (44) for easy input and readability. A front overlay (46) with tactile electronic switches allow easy input from user to respond to software driven menu commands. LED and audible alarms (48) provide notification to user of battery life and usage. A Leur-type lock connector (50) or delivery outlet establishes communication with the delivery line to either the nasal cannula device (52) shown in FIG. 2 or the inlet conduit on the modified Fruman valve (54) shown in FIG. 3.

More particularly, referring to FIG. 1b, the main unit (40) houses several main components. A first component or subassembly is comprised of an electronic/control portion of the device. It includes a microprocessor driven proportional valve or valve system (56), an alarm system, an electronic surveillance system and data input/output display system and electronic/software watch dog unit (44).

A second component or subassembly includes one or more disposable nitric oxide substrate cartridges (58) and an interface mechanism. A substrate converter system or segment (60) processes the primary compounds and converts it into pure nitric oxide gas. The gas then flows into an accumulator stable (62) and is regulated by the proportional valve assembly (56) into a NO outlet nipple (64).

A third component or subassembly is comprised of a secondary or backup nitric oxide system (66). It consists of mini-cylinders of high nitric oxide concentration under low-pressure. This system (66) is activated if and when the primary nitric oxide source (58) is found faulty, depleted or not available.

Nasal Cannula Adjunct

Referring to FIG. 2, there is shown a detailed drawing of a preferred embodiment of a valve (68) used to control the delivery of nitric oxide in a preset dosage through a disposable nasal cannula device (52) as shown. The valve (68) is controlled by the natural action of spontaneous respiration by the patient and the dosage is preset by the physical configuration of the device (52).

The device (52) including the valve (68) is constructed of dual lumen tubing (70). The internal diameter of the tubing (70) depends on the required dosage. The tubing (70) is constructed of material compatible with dry nitric oxide gas for the duration of the prescribed therapy. This tubing (70) is glued into the nasal cannula port (72).

The valve (68) is preferably comprised of a flexible flapper (74) that is attached by any mechanism, preferably a spot of adhesive (76), so as to be positioned over the supply tube (70). The flapper (74) must be sufficiently flexible to permit the valve action to be effected by the natural respiration of the patient. When the patient breathes in, the lower pressure in the nasal cannula device (52) causes the flapper (74) of the valve (68) to open and the dry gas is delivered from a reservoir (78) past the flapper (74) and into the patient's respiratory tract. When the patient exhales, positive pressure in the nasal cannula device (52) forces the flapper (74) of the valve (68) closed preventing any delivered gas entering the respiratory tract.

The supplied gas is delivered at a constant rate through the supply tube (70). The rate must be above that required to deliver the necessary concentration to the patient by filling the supply reservoir (78) up to an exhaust port (80) in the supply tube (70) during expiration. When the patient is exhaling the flapper (74) is closed and the supply gas feeds from a supply line (82) through a cross port (84) into the reservoir or storage chamber (78). The length of the reservoir chamber (78) given as dimension (86) determines the volume of gas delivered when the patient inhales. Inhaling opens the flapper (74) of the valve (68) and causes the reservoir chamber (78) to be emptied.

During exhalation when the flapper (74) is closed and the reservoir chamber (78) is filling, any excess gas exhausts through the exhaust port (80). During inhalation when the reservoir chamber (78) is emptied, the reservoir chamber (78) is displaced with atmospheric air through the exhaust port (80). There will continue to be supply gas from the supply line (82) through the cross port (84) during inhalation and this amount must be figured into the total delivered gas to determine the actual dosage. The tubing lumens (70) include various plugs (88) to direct the flow.

Mask/Valve Adjunct

Referring to FIG. 3, there is shown a further embodiment of a nitric oxide valve (54) which is a modification and improvement of a non-rebreathing valve for gas administration, referred to as a “Modified Fruman Valve,” as shown and particularly described in United States of America Patent No. 3,036,584 issued May 29, 1962 to Lee.

More particularly, the within invention specifically redesigns the Modified Fruman Valve for use in inhaled nitric oxide therapy. Specifically, in the preferred embodiment shown in FIG. 3, one end of a valve body (90) or valve body chamber is comprised of or includes a mask or mouth-piece (not shown) attached thereto. The connection is preferably standardized to a 22 mm O.D. to facilitate the attachment of the mask or mouth-piece. The other end of the valve body (90) is comprised of or provides an exhaust port (92). The exhaust port (92) entrains ambient air during the latter portion of inspiration and dilutes the nitric oxide coming from an inlet conduit (94).

The resultant nitric oxide concentration in the valve body (90) is determined by the dilutional factors regulated by the valve (54), tidal volume and the nitric oxide concentration in an attached flexed bag (96), being a fixed reservoir bag. The inlet conduit (94) is preferably spliced for the attachment of the small flexed bag (96). The purpose of the bag (96) is to act as a reservoir for nitric oxide gas. Further, an opening of the inlet conduit (94) is preferably modified to facilitate the attachment or connection of the inlet conduit (94) to a supply hose emanating from a nitric oxide supply chamber. Specifically, the opening of the inlet conduit (94) is preferably comprised of a knurled hose barb connector (98).

High Dosage gNO Administration

Although gaseous nitric oxide has been studied in connection with several applications as discussed in the background section, gNO has mainly been administered at low concentrations, such as about 200 ppm. Such low concentration was thought to be safely administered to mammals. Exposure times at about 200 ppm gaseous nitric oxide generally have been around the order of 30 minutes to several hours.

It has been found that administering high dosages of nitric oxide gas, such as between 1000 ppm and 50,000 ppm, may eradicate cancer cells, biofilms, and other pathogens or microbes, such as bacteria, mycobacteria, viruses and fungi. High dosages of nitric oxide gas preferably are about 10,000 ppm to about 50,000 ppm, more preferably about 20,000 ppm. Such high dosages are safely administered to mammals if the exposure times are limited to about 1 minute to 10 minutes. Exposure times preferably are about 3 minutes. The healthy or host cells or tissue have shown that they can survive such high dosages at these exposure times, while simultaneously eradicating cancer cells, biofilms, and other pathogens or microbes, such as bacteria, mycobacteria, viruses and fungi.

Different conditions which can be treated by high dosage administration of gNO include but are not limited to: topical treatments with gNO, cosmetic applications of gNO, vasodilation conditions with gNO, inhalation treatments with gNO, treatments of the blood with gNO, treatment of the skin or tissue with gNO, treatment of infections with gNO, treatment of inflammation on or within the body, and treatments of biofilms with gNO. Other conditions, aliments, or symptoms that may be treated with high dosage gNO include bronchoconstriction, reversible pulmonary vasoconstriction, asthma, pulmonary hypertension, adult respiratory distress syndrome (ARDS), and persistent pulmonary hypertension of the newborn (PPHN). Topical treatments may include treatment of wounds or surface infections. The present invention is thus not limited by the type of treatment that is contemplated.

The high dosage of gNO may be administered to mammals or to surfaces. Surfaces on or within the body, on medical devices, within hospitals and facilities, and the like may have or be susceptible to biofilms. The use of gNO to prevent and eradicate biofilms is described in U.S. application Ser. Nos. 10/953,827 and 11/592,950, herein incorporated by reference. Gaseous nitric oxide at high doses may be used to kill or prevent biofilms and also to sterilize surfaces in all the manners described in the above referenced applications.

The high dose gaseous nitric oxide may be delivered to the lungs and other areas of the respiratory tract through inhalation and non-inhalation methods. In non-inhalation methods, the gaseous nitric oxide may be delivered through an incubation tube. There is no need to time the delivery with the breathing of the mammal.

One or more treatments, delivery methods, delivery devices, and applications described in the following patents and patent applications, each herein incorporated by reference in its entirety, may benefit from the high dosage administration of gNO: U.S. Ser. No. 11/497,557; PCT/US05/016428; 11/445,965; 11/211,055; 11/591,373; 10/615,546; 10/658/665; 11/445,965; 11/066,790; 10/953,827; 11/592,950; 10/315,539; 11/158,902; 10/269,738; PCT/US05/016427; 11/021,109; PCT/US05/047319; PCT/IB06/0003265; 60/810,938; 11/107,618; PCT/US06/14414; 10/615,546; 11/591,373; 6,920,876; 7,122,018; 5,485,827; 5,873,359; 6,432,077; and 6,793,644.

For anti-inflammatory applications of high concentrations of gNO, it is hypothesized that the high dose of NO acts as a feedback loop to down regulate iNOS, and/or acts as a antioxidant on peroxynitrite and other reactive oxygen species and/or remove the stimulus such as bacterial load all leading to the amelioration of high levels of NO or its reactants resulting in damaging pathogenesis. This hypothesis is supported by testing results of the Applicant wherein 200 ppm gNO was topically applied to an infected wound on the skin. The bacterial load was significantly reduced, histological results showed a significant reduction in inflammatory bodies and the serum NOX levels were lower as compared to the infected control. It is hypothesized that the body stopped producing the “bad” NO causing pathogenic inflammation.

While the art has shown that while low concentrations (about 200 ppm) of gNO can be bathed over tissue and surfaces, high concentrations (about 1000 ppm to 50,000 ppm) may require localized administration of gNO, so as to minimize the exposure of healthy collateral host cells to the gNO. The high dosages of gNO may be applied to the targeted areas in a manner similar to those described herein.

In another application of high concentrations (about 1000 ppm to 50,000 ppm) of gNO, gNO may be directed to non-cancerous or “healthy” skin. Such high concentrations are expected to kill the skin to a certain level, but such damage is tolerable and wanted. The damage that is anticipated is similar to the damage to the skin associated with chemical peels, acid rinses, certain tattoo removal treatments, and other similar treatments that involve an intentional step of damaging the skin cells. The damaging of the skin initiates, accelerates, facilitates, and necessitates the new skin growth. In these applications the high dosages of gNO may be delivered to the mammal for time periods not limited to about 1 to 10 minutes. In these applications, time periods of longer than 10 minutes may be tolerable since damage to the host cells is anticipated.

High concentrations of gNO may be directed to a large general area of the skin, such as the face. In these applications, damage to the top layer of skin cells is anticipated, but this will accelerate or stimulate new skin growth. The removal of an outer layer of skin will reveal a younger newer layer. These applications may be thought of as one type of cosmetic application of high concentrations of gNO.

High concentrations of gNO may be directed to a targeted area of the skin, in a controlled application. The devices disclosed below may be suitable for the delivery of the gNO. The targeted area of the skin may be a mole, liver spot, wart, skin tag, tattoo, or any other undesirable area of the skin.

High concentration gNO may be delivered through an inhaler, such as a typical asthma inhaler. The gNO may be the carrier gas or the inhaler may use aerosolized particles of nitric oxide releasing compounds with a carrier gas. In either case, the concentration of the gaseous nitric oxide is between 1000 ppm and 50,000 ppm.

The inhaler may use NO-releasing, NO-donor, or NO-upregulator compounds. For simplicity, NO-releasing, NO-donor and NO-upregulators will be referred to only as “NO-releasing compounds.” Known NO-releasing compounds useful in the methods and devices of the invention include, but are not limited to: nitroso or nitrosyl compounds characterized by an —NO moiety that is spontaneously released or otherwise transferred from the compound under physiological conditions (e.g. S-nitroso-N-acetylpenicillamine, S-nitroso-L-cysteine, nitrosoguanidine, S-nitrosothiol, and others described in WO 92/17445 and U.S. Pat. No. 5,427,797 (herein incorporated by reference)). In addition, other NO-releasing compounds include compounds in which NO is a ligand on a transition metal complex, and as such is readily released or transferred from the compound under physiological conditions (e.g. nitroprusside, NO-ferredoxin, NO-heme complex) and nitrogen-containing compounds which are metabolized by enzymes endogenous to the respiratory and/or vascular system to produce the NO radical (e.g. arginine, glyceryl trinitrate, isoamyl nitrite, inorganic nitrite, azide and hydroxylamine). More NO-releasing compounds are polyethyleneimine (PEI)-based polymers exposed to NO gas; molsidomine; nitrate esters; sodium nitrite; iso-sorbide didinitrate; penta erythritol tetranitrate; nitroimidazoles; complexes of nitric oxide and polyamines; anionic diazeniumdiolates (NONOnates) (including those disclosed in U.S. Pat. Nos. 4,954,526 and 5,155,137) and the NO releasing compounds disclosed in U.S. Pat. No. 5,840,759 and PCT WO 95/09612. Examples of NONOate compounds include diethylamine/NONO, diethylenetriamine/NONO, and methylaminohexylmethylamine/NONO (illustrated in Hanson et al., Nitric Oxide, Biochemistry, Molecular Biology, and Therapeutic Implications, Ignarro and Murad, Ed., Academic Press, New York (1995)). An NO-releasing compound, donor or upregulator can be provided in powder form or as a liquid (e.g., by mixing the compound with a biologically-compatible excipient).

The NO-releasing compound may be administered to a patient alone or in conjunction with NO gas, CO gas, a carrier gas or another NO-releasing compound. When more than one compound is administered to the patient, the compounds can be mixed together, or they can be administered to the patient sequentially. Any one, or a combination, of the following routes of administration can be used to administer the NO-releasing compound(s) to the patient: intravenous injection, intraarterial injection, transcutaneous delivery, oral delivery, and inhalation (e.g., of a gas, powder or liquid).

The NO-releasing compound selected for use in the method of the invention may be administered as a powder (i.e., a finely divided solid, either provided pure or as a mixture with a biologically-compatible carrier powder, or with one or more additional therapeutic compounds) or as a liquid (i.e., dissolved or suspended in a biologically-compatible liquid carrier, optionally mixed with one or more additional therapeutic compounds), and can conveniently be inhaled in aerosolized form (preferably including particles or droplets having a diameter of less than 10 μm). Carrier liquids and powders that are suitable for inhalation are commonly used in traditional asthma inhalation therapeutics, and thus are well known to those who develop such therapeutics. The optimal dosage range can be determined by routine procedures by a pharmacologist of ordinary skill in the art. For example, a useful dosage level for SNAP would be from 1 to 500 μmoles (preferably 1-200 μmoles) per inhaled dose, with the number of inhalations necessary varying with the needs of the patient.

When an NO-releasing compound is inhaled in solid or liquid form, the particles or droplets are deposited throughout the respiratory system, with larger particles or droplets tending to be deposited near the point of entry (i.e., in the mouth or nose) and smaller particles or droplets being carried progressively further into the respiratory system before being deposited in the trachea, bronchi, and finally the alveoli. (See, e.g., Hounam & Morgan, “Particle Deposition”, Ch. 5 in Respiratory Defense Mechanisms, Part 1, Marcel Dekker, Inc., NY; ed. Brain et al., 1977; p. 125.) A particle/droplet diameter of 10 mu or less is recommended for use in the method of the invention. Determination of the preferred carrier (if any), propellant (which may include NO diluted in an inert gas such as N₂), design of the inhaler, and formulation of the NO-releasing compound in its carrier are well within the abilities of those of ordinary skill in the art of devising routine asthma inhalation therapies. The portable inhaler could contain an NO-releasing compound either mixed in dry form with a propellant or held in a chamber separate from the propellant, or mixed with a liquid carrier capable of being nebulized to an appropriate droplet size, or in any other configuration known to those skilled in portable inhaler technology. A few of the several types of inhaler designs that have been developed to date are discussed in, for example, U.S. Pat. Nos. 4,667,668; 4,592,348; 4,534,343; and 4,852,561, each of which patents is herein incorporated by reference. Other inhaler designs are described in the Physicians'Desk Reference, 45th Edition, Edward R. Barnhart, Publisher (1991). Each of these and other aerosol-type inhalers can be adapted to accommodate the delivery of NO-releasing compounds. Also useful for delivering an NO-releasing compound formulated in dry powder form is a non-aerosol-type inhaler device such as that developed by Allen & Hanburys, Research Triangle Park, N.C.

Delivery of “Pure” gNO

Another embodiment of the present invention is the application of about 1% to about 100% gNO (10,000 ppm to 1,000,000 ppm). About 1% to about 100% gNO may be applied to a targeted surface through the use of a needle or similar delivery device. The delivery may be through a needle or an array of needles, such as nano-sized, micron-sized, and similarly sized needles. With the delivery of gNO through a needle, very small surfaces on or within the body or on another surface may be targeted. The gNO at very high concentration may be delivered at very small volumes. Such small volumes would be allowed to be absorbed into the surface, such as the skin of a mammal. gNO may be delivered to a small tumor or to a small skin imperfection, such as a wart.

Tumorcidal Activity of Gaseous NO

Methods provided herein may be useful for the treatment, control, or prevention of growths of cancerous cell phenotype in animal bodies, and preferably in mammalian bodies, and more preferably in human bodies. Types of cancerous growths that may be treated, controlled, or prevented by use of the methods herein include, but are not limited to, benign tumors including hemangiomas, acoustic neuromas, neurofibroma, trachomas and pyogenic granulomas; malignant tumors; and metastasis. Cancerous cell phenotypes that may be treated, controlled, or prevented by use of the methods herein include, but are not limited to, adenocarcinomas; central nervous system cancers including brain cancer; cervical cancers; cholangiocarcinomas; colon cancers; colorectal cancers; erythroleukemias; gastric sarcomas; gliomas; head and neck cancers; intestinal cancers; lung cancers including small cell lung cancers and non-small cell lung cancers; lymphomas; melanomas; multiple myelomas; osteosarcomas; ovarian cancers; pancreatic cancers; prostrate cancers; sarcomas; stomach cancers; testicular cancers; and uterine cancers. Cancers generally located in any location in or on an animal body, and more preferably a human body, may be treated, controlled, or prevented by use of the methods herein including, but not limited to, cancers located in or on the adrenal gland, bladder, bones, brain, breast, cervix, colon, colorectum, esophagus, gastrointestinal tract, heart, kidney, liver, large intestine, lungs, mouth, ovaries, pancreas, parathyroid, pituitary gland, prostate, salivary gland, skin, small intestine, spleen, stomach, thymus, thyroid, testicles, urinary tract, uterus, vagina, and so forth.

One of skill in the art will appreciate that the methods provided herein for treating, controlling, or preventing cancer may be generally applicable to all know or to-be-discovered cancerous cell phenotypes and cancerous growths.

Methods provided herein may be especially useful for the treatment, control, or prevention of tumors at localized sites including inoperable tumors or in tumors where localized treatment would be beneficial, and solid tumors.

Cancer cells are similar to host cells with regard to their detoxification thiol pathways. gNO acts to inhibit growth and or cause cell death by binding available intracellular thiols that normally protect the cell or microbe by detoxifying electrophiles. Without thiols to protect the cell or microbe, the gNO and hydrogen peroxide molecules are free to cause rapid intracellular damage such as but not limited to deamination of DNA, reacting with enzymes to release metal ions, and reacting with oxygen to create highly cytotoxic compounds like peroxynitrite. Similarly, the pathway of killing cancerous phenotypes and growths is analogous to the pathway of killing pathogens, such as bacteria based pathogens, as described in the parent application.

Accordingly, there is provided a method for the treatment, control, or prevention of cancerous cell phenotypes and growths in an animal. The method comprises providing gaseous nitric oxide, administering the gaseous nitric oxide to the mammal at one or more administration sites, and optionally scavenging excess gaseous nitric oxide from the one or more administration sites. Preferably, the animal being treated by the method described herein is a mammal, and more preferably is a human.

The gNO may be provided by an external source, for example a reservoir of gNO or a chemical generator of gNO. For example, the nitric oxide source may be any nitric oxide producing compound, composition or substance. In other words, the nitric oxide source may be any compound, composition or substance capable of producing or providing nitric oxide, and particularly, nitric oxide gas. For instance, the compound, composition or substance may undergo a thermal, chemical, ultrasonic, electrochemical or other reaction, or a combination of such reactions, to produce or provide gaseous nitric oxide which is to be administered for the treatment, control, or prevention of growths of cancerous cell phenotypes in accordance with the methods described herein.

The gNO may be of medical purity, that is, preferably at least about 95%, more preferably at least about 99%, and even more preferably at least about 99.5% pure gNO. More preferably, however, the gNO for use in the methods for the treatment, control, or prevention of cancerous cell phenotypes and growths disclosed herein may be a mixture of gNO and other gases, preferably gases such as air, nitrogen, oxygen, and so forth. For example, the gNO may be administered in a concentration of about 200 ppm, or of about 350 ppm, or of greater than about 350 ppm. Varying the concentration of gNO that is administered in accordance with the methods described herein may alter the efficacy of the method in treating, controlling, or preventing the growth of cancerous cell phenotypes. For example, in some instances cancer cells may exhibit greater than 90% survival after exposure to 200 ppm gNO for 24 hours. However, cancer cells may be more susceptible to higher doses of gNO, for example, exposure to 350 ppm gNO exposure for 24 hours. Varying the concentration of gNO that is administered in accordance with the methods described herein also may alter the detrimental effects, if any, upon adjacent healthy tissues. Accordingly, the concentration of gNO that is administered may be varied in order to optimize the lethality of the gNO to cancerous cell phenotypes compared to the lethality to normal cell phenotypes. Preferably, the gNO mixture is sanitized in order to reduce or prevent the inadvertent spread of infectious agents during administration of the mixture.

The gNO may be administered for varying periods of time. For example, when gNO is administered at a concentration of about 200 ppm, the administration may last for about 1 hour, or for about 2.5 hours, or for about 3.5 hours. When gNO is administered at a concentration of about 350 ppm, the administration may last for about 2 hours, or for at least about 2 hours. It will be appreciated that the time of administration may be varied according to the concentration of gNO that is to be administered, the type and location of the cancerous growth(s) that are to be treated, the particularities of the animal, mammal, or human that is to be treated, and so forth.

gNO Delivery Devices

Some microbial diseases, like tuberculosis, reside in loculated areas where gaseous gNO cannot penetrate. Use of high-dose gNO to treat cancer cells and loculated microbes requires localized delivery techniques.

There are no devices available to deliver gNO directly to target area without causing collateral host cells damage. Accordingly, bronchoscopes and endoscopes modified to accommodate a gNO delivery system are described herein. Early tumor detection technologies exist but there are no methods to treat and evaluate the effectiveness of the treatment at the same time. Accordingly, a new device that includes a gNO delivery system inside a blue-fluorescence bronchoscope/endoscope is described herein. At least one way in which the described devices are useful is that grade 1 and 2 cancer cells may be identified, treated, and evaluated during the same procedure.

Another embodiment of the invention is a method of delivering gaseous nitric oxide to a mammal comprising: identifying cancerous or abnormal cells inside the mammal with a device and administering the gaseous nitric oxide to the identified cancerous or abnormal cells with the same device. The gaseous nitric oxide may be at lower traditional dosages or at high concentrations ranging from about 1000 ppm to about 50,000 ppm.

Generally, devices that may be useful for localized delivery of gNO to target cells with limited exposure to surrounding healthy host cells may comprise a small bore inner cannula that delivers an adequate dose of gNO to the target site. Preferably the target site is from about 1 mm to about 2 cm² in size. The device further may comprise an outer lumen through which a sufficient vacuum may be applied to scavenge excess gNO away from host cells and tissue that surround and border the target site. In this way gNO may be delivered to the target site, preferably without excessive damage to healthy host tissues.

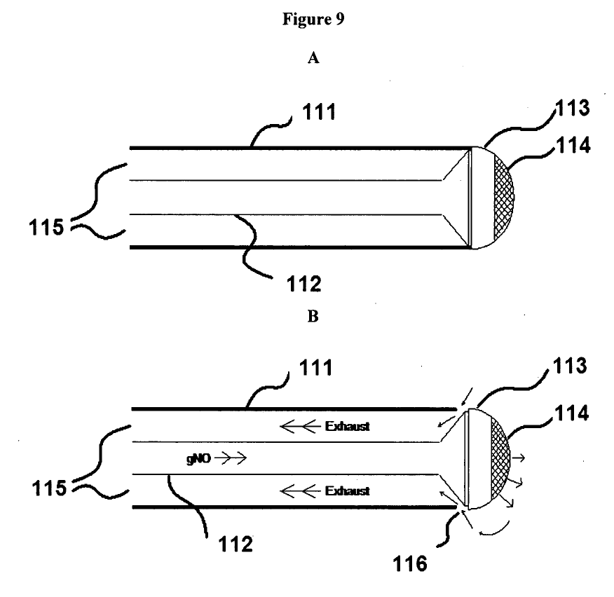

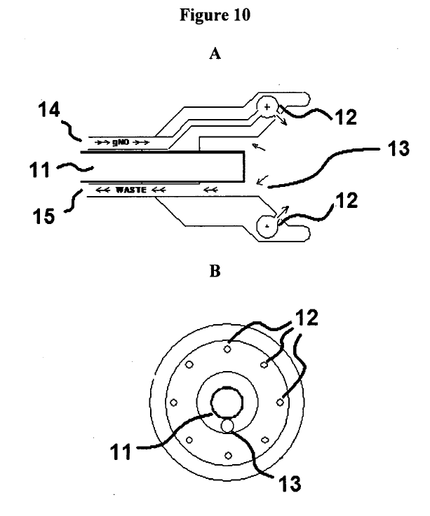

FIGS. 9 and 10 illustrate exemplary devices that may be used to deliver gNO to administration sites of animal bodies, and more preferably mammalian bodies, and even more preferably human bodies, for the treatment purposes described herein. The devices may function by delivering gNO to one or more administration sites by a positive pressure gradient, and scavenging gNO from the one or more administration sites by a negative pressure gradient. In this way, the devices may function to deliver gNO to one or more administration sites without causing excessive damage to collateral host cells because the gNO gas that is administered preferably is removed from the one or more administration sites before collateral cells suffer excessive damage. It is to be noted, however, that a certain level of damage to the host cells may be tolerated, and that the conditions under which the gNO gas is administered may be optimized to decrease damage to collateral cells while also providing the preferred therapeutic effects described herein. Damage to the host cells to a certain level is acceptable and no where suggested in the prior art.

In FIG. 9, embodiments A and B, a device to deliver gNO from one or more administration sites comprises an outer lumen 111 or cannula, trocar, tube, etc. Disposed coaxially inside of the outer lumen 111 is an inner lumen 112 or cannula, tube, etc. In FIG. 9, the inner lumen 112 is disposed approximately centrally in the outer lumen 111; however, one of skill in the art will appreciate variations of this design in accordance with the description herein.

A space 115, preferably an exhaust space, is between the outer lumen 111 and inner lumen 112. Although illustrated as an annular space, it will be appreciated that the exhaust space 115 could take any number of different geometries dependant upon the shape and configuration of the outer lumen 111 and inner lumen 112. Attached to the inner lumen 112 at the distal end is a tip 113 in fluid communication with the inner lumen 112. The tip 113 comprises a wire mesh or screen 114 that accesses the space inside of the inner lumen 112. The delivery device may be advanced to an administration site in the retracted configuration shown in FIG. 9, embodiment A. Preferably, as illustrated, the tip 113 is rounded and seals the outer lumen 111 when it is in the retracted position, thus easing insertion of the tip into the body where it is brought adjacent to an administration site.

When the tip 113 of the device is brought adjacent to an administration site, the device is adjusted to its extended configuration in order to affect the administration and scavenging of gNO. In FIG. 9, embodiment B, the device is illustrated in the extended configuration wherein the tip 113 is extended beyond the distal end of the outer lumen 111. In the extended configuration, an exhaust path 116 is opened between the distal end of the outer lumen 111 and the tip 113. gNO is delivered through the inner lumen 112. The gNO exits the inner lumen 112 at the tip 113 that is in fluid communication with the inner lumen 112. The wire mesh or screen 114 at the distal end of the tip 113 may assist in diffusing the gNO gas as it exists the device. The exhausted gNO gas returns to the device at the exhaust path 116. A vacuum is applied to the exhaust space 115 between the outer lumen 111 and the inner lumen 112 in order to attract the exhausted gNO. The exhausted gNO then is brought through the exhaust space 115 to exit the body and be disposed of appropriately. In this way, the device is capable of scavenging gNO from an administration site.

In an alternative configuration of the device illustrated in FIG. 9, the flow of gNO could be reversed so that gNO is delivered through the space between the outer lumen and inner lumen and removed from the administration site through the inner lumen. In this alternative configuration, a vacuum would be applied to the inner lumen and a positive pressure of gNO would be applied to the space defined by the inner and outer lumens. In another alternative, the tip is permanently secured to the outer lumen as well as the inner lumen, such that the tip does not have retracted and extended configurations. In this alternative, permanent passages are provided in the outer lumen for gNO to be expelled from the device or sucked into the device by vacuum. For example, the permanent passages could be small holes or slits radially disposed around the of the outer lumen, preferably near the distal end of the outer lumen so as to be near the tip of the device.

The device depicted in FIG. 9 preferably could be attached to the end of an endoscope or bronchoscope (e.g. a blue-fluorescence endoscope or bronchoscope) so that the insertion of the device into the body, advancement towards the administration site, and retraction from the body could be visually observed by or otherwise made known to the operator or someone working in concert with the operator. Alternatively, the device depicted in FIG. 9 could be attached to a guidewire in order to more effectively insert, advance, and retract the device. Additionally, the device depicted in FIG. 9 could be coated in a fluoroscopic material or have one or more fluoroscopic tags attached to it so that its insertion, advancement, and retraction could be fluoroscopically observed.

In FIG. 10, embodiments A and B, another device to deliver and scavenge gNO from one or more administration sites is illustrated. The device is intended to fit over the distal end or tip of an endoscope or bronchoscope 11. The device is annular-shaped, meaning that it has a hole in its center or about in its center and is approximately circular in shape. The hole about in the center of the annular-shaped device is sized to accommodate the distal end of an endoscope or bronchoscope. For example, in a preferred configuration the annular-shaped device has a hole in about its center, wherein the hole is about 0.5 cm to about 1.5 cm in diameter. Preferably the hole is sized so as to fit snugly over the distal end of an endoscope or bronchoscope.

The device comprises a gas supply passage 14 in fluid communication with gas supply openings 12. The device further comprises an exhaust passage 15 in fluid communication with an exhaust opening 13. It will be appreciated that, in alternative configurations of this device, one or more gas supply openings 12 and one or more exhaust openings 13 may be provided in the device, and that one or more gas supply passages 14 and one or more exhaust passages 15 also may be provided in the device, in accordance with the description herein. The device may be inserted into a body and advanced adjacent to an administration site. When the device is appropriately positioned, gNO is supplied to the gas supply passage 14 and exits the device through the gas supply openings 12. Vacuum is applied to the exhaust passage 15 so that gNO is attracted to the exhaust opening 13 and removed from the administration site. In this way, the device is capable of scavenging gNO from an administration site. The lip 16 of the device preferably extends beyond the distal end of the endoscope or bronchoscope 11 and fits against a tumor in order to form a seal, thus further reducing damage caused by the gNO to adjacent normal cells that are outside of the area sealed off by the lip 16 of the device.

In some instances, devices for localized gNO delivery and scavenging may not be required in order to administer the gNO in accordance with the methods described herein. For example, in the case of some lung cancers, gNO may be delivered via inhalation. Non-inhalation direct deliverance methods may be used as well. Preferably, the concentration of gNO and time of administration may be adjusted to minimize, or at least optimize, the systemic effects of gNO administration to the subject (e.g. animal, mammal, or human) of the treatment. In the case of some skin cancers, gNO may be delivered topically to one or more administration sites by use of devices available in the art for the topical administration of gases. Again, the effect of gNO on adjacent healthy cells may be minimized or at least optimized by adjusting the concentration of gNO administered topically and the time of administration. One skilled in the art will appreciate in general how gNO may be administered topically or by inhalation routines.

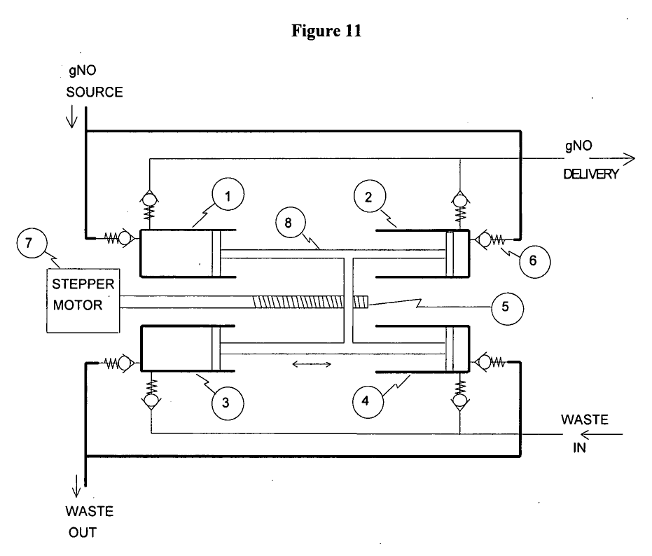

The gNO may be delivered to the delivery devices described herein from a tank of gNO or other positive-pressure source. Alternatively, the localized delivery devices described herein may be attached to a pumping mechanism for delivering gNO to the localized delivery devices. Described herein is an exemplary pumping mechanism illustrated in FIG. 11. The pumping mechanism comprises a 4-cylinder pump consisting of 2 pairs of opposed cylinders.

One cylinder (1, 2) on each side of the pumping mechanism pumps gNO and one cylinder (3, 4) on each side extracts waste gNO by creating a vacuum. The pump is controlled by a stepper or servo motor (7) to provide variable speed and stroke of the piston assembly (8) to allow for precise control of the volume of gas to be delivered. One possibility is to use a threaded rod (5) driven by the stepper motor to move the piston assembly (8) back and forth. Check valves (6) control the flow of gas. Gas flow may not be completely continuous as there would be a slight cessation of flow as the pistons reverse direction at the end of each stroke. Appropriate filtering and fluid traps may be required on the delivery tubing and waste collection tubing between the pump assembly and the delivery device.

The methods and devices of the invention will now be described in greater detail in the following examples.

### EXAMPLES

### Example 1

The purpose of this example is to illustrate the effect of continuous gaseous nitric oxide

(gNO) 200 ppm and 350 ppm compared to air (control) on the survival of a representative line of cancer cells. Cancer cell lines (A549-epithelial lung carcinoma cells and H460-epithelial metastatic; large cell lung cancer cells) were prepared in F12 medium and suspended in six 96-well plates (3 treatment and 3 controls). The plates were incubated in air (control), 200 ppm gNO, or 350 ppm gNO for 24 hours. The plates then were removed and cell viability assessed by an MTS proliferation assay.

Results showed greater than 90% survivability (A549) in 200 ppm gNO whereas there was only a 1% (A549) and a 2% (H460) survivability in 350 ppm gNO after 24 hours of continuous exposure as compared to the cells exposed to air (control).

Tables 1, 2, and 3 below demonstrate the survivability of A549 cells in Ham's F12 and Hank's Balanced Salt Solution (HBSS) mediums (both commercially available from Invitrogen Gibco-BRL, Burlington, Ontario, Canada) when exposed to 200 ppm gNO for, respectively, 1 hour, 2.5 hours, and 3.5 hours.

TABLE 1
A549 CELLS EXPOSED TO 200 PPM NO FOR 1 HOUR
IN HAM'S F12 AND HANK'S
BALANCED SALT SOLUTION (HBSS) MEDIUMS

| | F12 medium | | | HBSS medium | | |
|---|---|---|---|---|---|---|
| | NO | Control | Blank | NO | Control | Blank |
| | 0.945 | 1.311 | 0.297 | 0.067 | 0.493 | 0.062 |
| | 0.886 | 1.099 | 0.23 | 0.062 | 0.375 | 0.064 |
| | 0.842 | 1.151 | 0.23 | 0.062 | 0.369 | 0.061 |
| | 0.955 | 0.851 | 0.226 | 0.058 | 0.35 | 0.059 |
| | 0.793 | 0.91 | 0.235 | 0.057 | 0.293 | 0.059 |
| | 0.818 | 0.959 | 0.212 | 0.058 | 0.31 | 0.057 |
| | 0.828 | 0.713 | 0.211 | 0.056 | 0.275 | 0.06 |
| | 0.78 | 0.764 | 0.208 | 0.06 | 0.297 | 0.056 |
| | 0.805 | 0.995 | | 0.06 | 0.404 | |
| | 0.936 | 0.848 | | 0.06 | 0.342 | |
| | 0.959 | 0.682 | | 0.067 | 0.313 | |
| | 0.91 | 0.66 | | 0.059 | 0.257 | |
| | 0.827 | 0.603 | | 0.057 | 0.294 | |
| | 0.8 | 1.125 | | 0.056 | 0.385 | |
| | 0.795 | 0.888 | | 0.055 | 0.341 | |
| | 0.759 | 0.896 | | | 0.361 | |
| Average | 0.852 | 0.903 | 0.231 | 0.06 | 0.341 | 0.06 |
| Corrected Average | 0.621 | 0.672 | | 0 | 0.281 | |
| STDEV | 0.068 | 0.198 | 0.029 | 0.004 | 0.058 | 0.003 |
| Survival | 92% | | | 0% | | |

TABLE 2
A549 CELLS EXPOSED TO 200 PPM NO FOR 2.5 HOURS
IN HAM'S F12 AND HANK'S
BALANCED SALT SOLUTION (HBSS) MEDIUMS

| | F12 medium | | | HBSS medium | | |
|---|---|---|---|---|---|---|
| | NO | Control | Blank | NO | Control | Blank |
| | 1.284 | 1.682 | 0.385 | 0.069 | 0.733 | 0.067 |
| | 1.299 | 1.497 | 0.355 | 0.062 | 0.57 | 0.071 |
| | 1.261 | 1.452 | 0.357 | 0.063 | 0.532 | 0.067 |
| | 1.178 | 1.142 | 0.345 | 0.059 | 0.502 | 0.065 |
| | 1.33 | 1.115 | 0.348 | 0.058 | 0.417 | 0.063 |
| | 1.422 | 1.374 | 0.342 | 0.059 | 0.447 | 0.06 |
| | 1.454 | 1.764 | 0.37 | 0.058 | 0.396 | 0.064 |
| | 1.458 | 1.465 | | 0.061 | 0.409 | 0.06 |
| | 1.354 | 1.155 | | 0.06 | 0.616 | |
| | 1.105 | 1.094 | | 0.06 | 0.554 | |
| | 1.348 | 1.192 | | 0.069 | 0.508 | |
| | 1.082 | 1.656 | | 0.059 | 0.413 | |
| | 1.329 | 1.292 | | 0.057 | 0.461 | |
| | 1.237 | 1.425 | | 0.057 | 0.624 | |
| | 1.272 | | | 0.056 | 0.522 | |
| | 0.995 | | | | 0.527 | |
| Average | 1.276 | 1.379 | 0.357 | 0.06 | 0.514 | 0.065 |
| Corrected Average | 0.918 | 1.02 | | 0 | 0.450 | |
| STDEV | 0.132 | 0.223 | 0.015 | 0.004 | 0.092 | 0.004 |
| Survival | 90% | | | 0% | | |

TABLE 3
A549 CELLS EXPOSED TO 200 PPM NO FOR 3.5 HOURS
IN HAM'S F12 AND HANK'S
BALANCED SALT SOLUTION (HBSS) MEDIUMS

| | F12 medium | | | HBSS medium | | |
|---|---|---|---|---|---|---|
| | NO | Control | Blank | NO | Control | Blank |
| | 1.561 | 2.138 | 0.48 | 0.07 | 0.938 | 0.064 |
| | 1.625 | 1.947 | 0.453 | 0.063 | 0.718 | 0.062 |
| | 1.587 | 1.915 | 0.453 | 0.063 | 0.662 | 0.066 |
| | 1.525 | 1.493 | 0.46 | 0.059 | 0.623 | 0.062 |
| | 1.665 | 1.504 | 0.506 | 0.058 | 0.520 | 0.073 |
| | 1.444 | 1.839 | 0.474 | 0.059 | 0.552 | 0.074 |
| | 1.642 | 1.086 | 0.471 | 0.058 | 0.500 | 0.073 |
| | 1.332 | 0.913 | 0.463 | 0.061 | 0.496 | 0.071 |
| | 1.639 | 2.183 | | 0.061 | 0.731 | |
| | 1.551 | 1.775 | | 0.06 | 0.676 | |
| | 1.567 | 1.486 | | 0.069 | 0.603 | |
| | 1.863 | 1.433 | | 0.059 | 0.494 | |
| | 1.335 | 1.55 | | 0.058 | 0.571 | |
| | 1.325 | 2.219 | | 0.057 | 0.785 | |
| | 1.575 | 1.726 | | 0.056 | 0.650 | |
| | 1.227 | 1.856 | | | 0.654 | |
| Average | 1.529 | 1.691 | 0.47 | 0.061 | 0.636 | 0.068 |
| Corrected Average | 1.059 | 1.22 | | 0 | 0.568 | |
| STDEV | 0.161 | 0.371 | 0.017 | 0.004 | 0.12 | 0.005 |
| Survival | 87% | | | 0% | | |

As seen in Table 1, A549 cells have a survival rate of up to about 92% when exposed to 200 ppm gNO for 1 hour in F12 medium. As seen in Table 2, A549 cells have a survival rate of up to about 90% when exposed to 200 ppm gNO for 2.5 hours in F12 medium. As seen in Table 3, A549 cells have a survival rate of up to about 87% when exposed to 200 ppm gNO for 3.5 hours in F12 medium.

When exposed to 350 ppm of gNO, however, cancer cell survival rates decrease dramatically. Tables 4 and 5 below demonstrate the survivability of A549 and H460 cells in Ham's F12 and RMPI mediums (both commercially available from Invitrogen Gibco-BRL, Burlington, Ontario, Canada) when exposed to 350 ppm gNO for 2 hours.

This dramatic decrease in survival rates may be attributed to the difference in the mediums used. It is known that the presence of minerals and substrates in certain mediums may act to bind the NO molecules so the molecules are not otherwise available.

TABLE 4
A549 CELLS EXPOSED TO 350 PPM NO OR CONTROL
(AIR) FOR 2 HOURS IN F12 MEDIUM

| | NO | Blank | Control | Blank |
|---|---|---|---|---|
| | 0.106 | 0.133 | 1.472 | 0.113 |
| | 0.110 | 0.090 | 1.703 | 0.112 |
| | 0.112 | 0.090 | 1.676 | 0.109 |
| | 0.110 | 0.091 | 1.644 | 0.113 |
| | 0.104 | | 1.543 | |
| | 0.115 | | 1.637 | |
| | 0.106 | | 1.620 | |
| | 0.106 | | 1.575 | |
| | 0.110 | | 1.668 | |
| | 0.116 | | 1.580 | |
| | 0.119 | | 1.573 | |
| | 0.108 | | 1.548 | |
| | 0.104 | | 1.655 | |
| | 0.108 | | 1.490 | |
| | 0.107 | | 1.519 | |
| | 0.120 | | 1.544 | |
| Average | 0.110 | 0.101 | 1.590 | 0.112 |
| Corrected Average | 0.009 | | 1.478 | |
| STDEV | 0.005 | 0.021 | 0.069 | 0.002 |
| Survival | 1% | | | |

TABLE 5
H460 CELLS EXPOSED TO 350 PPM NO OR CONTROL
(AIR) FOR 2 HOURS IN RPMI MEDIUM

| | NO | Blank | Control | Blank |
|---|---|---|---|---|
| | 0.158 | 0.133 | 2.042 | 0.135 |
| | 0.145 | 0.118 | 1.925 | 0.133 |
| | 0.184 | 0.119 | 1.865 | 0.137 |
| | 0.143 | 0.113 | 1.837 | 0.133 |
| | 0.193 | | 1.992 | |
| | 0.147 | | 1.955 | |
| | 0.152 | | 1.859 | |
| | 0.154 | | 1.86 | |
| | 0.156 | | 1.798 | |
| | 0.172 | | 1.833 | |
| | 0.154 | | 1.768 | |
| | 0.157 | | 1.836 | |
| | 0.149 | | 1.767 | |
| | 0.165 | | 1.832 | |
| | 0.149 | | 1.85 | |
| | 0.155 | | 1.952 | |
| Average | 0.158 | 0.121 | 1.873 | 0.135 |
| Corrected Average | 0.037 | | 1.738 | |
| STDEV | 0.014 | 0.009 | 0.079 | 0.002 |
| Survival | 2% | | | |

As seen in Table 4, A549 cells have a survival rate of only about 2% when exposed to 350 ppm gNO for 2 hours. And as seen in Table 5, H460 cells have a survival rate of only about 0% when exposed to 350 ppm gNO for 2 hours. This data demonstrates that 350 ppm doses of gNO may have a high propensity to kill other cancerous cell phenotypes and growths in addition to the tested A549 and H460 lines.

### Example 2

The purpose of this example is to observe the possibility of a novel bactericidal high dose (25,000 ppm) effect of gaseous nitric oxide (gNO) on an ATCC strain of Staphylococcus aureus plated on a blood agar media and determine a time effect of this dosage.

A closed environment treatment chamber was used (see, “A direct nitric oxide gas delivery system for bacterial and mammalian cell cultures,” A. Ghaffari, D. H. Neil, A. Ardakani, J. Road, A. Ghahary, C. C. Miller. Nitric Oxide 12(3):129-140, 2005, herein incorporated by reference in its entirety). The following steps were performed:
1. Calibrated the AeroNOx analyzer as per standard procedure.
2. Calibrated the closed environment treatment chamber as per standard procedure.
3. In a 50 mL tube, known Staphylococcus aureus bacteria in 10 mL Nutrient Broth was grown overnight or for about 12 hours in a shaker incubator.
4. Measured optical density of grown Staphylococcus aureus using a spectrophotometer set at 600 nm. Used original nutrient broth as a blank. O.D. should be 1.0±10%.
5. Turned on closed environment treatment chamber.
6. Labeled three plates: Air, NO+1 minute, NO+3 minutes.
7. Immersed a sterile cotton swab into the bacterial mixture in the 50 mL tube and shaked off excess liquid.
8. Swiped one plate entirely in two directions with the swab.
9. Repeated steps 7-8 using the cotton swab for the remaining three plates.
10. Using clean technique, utilized the device to administer either air or 25,000 ppm gNO at a fixed distance and flow towards the inoculated agar media. The agar plate was enclosed in an isolated plastic box with lid. A micropipette through which the gas flowed was affixed to the box lid. The box lid was sealed through the use of plastic wrap. The micropipette was in fluid communication with the gaseous mixing manifold in order to deliver the specified concentrations.
11. Placed the three treated plates into the laboratory incubator.
12. Removed plates after 8 hours and placed in a 37° C. incubator for 12-16 hours.
13. After incubation, recorded results with digital pictures.
14. Sealed plates with paraffin and refrigerated for storage.

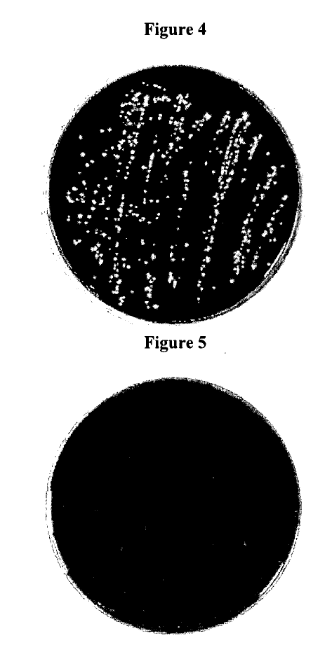

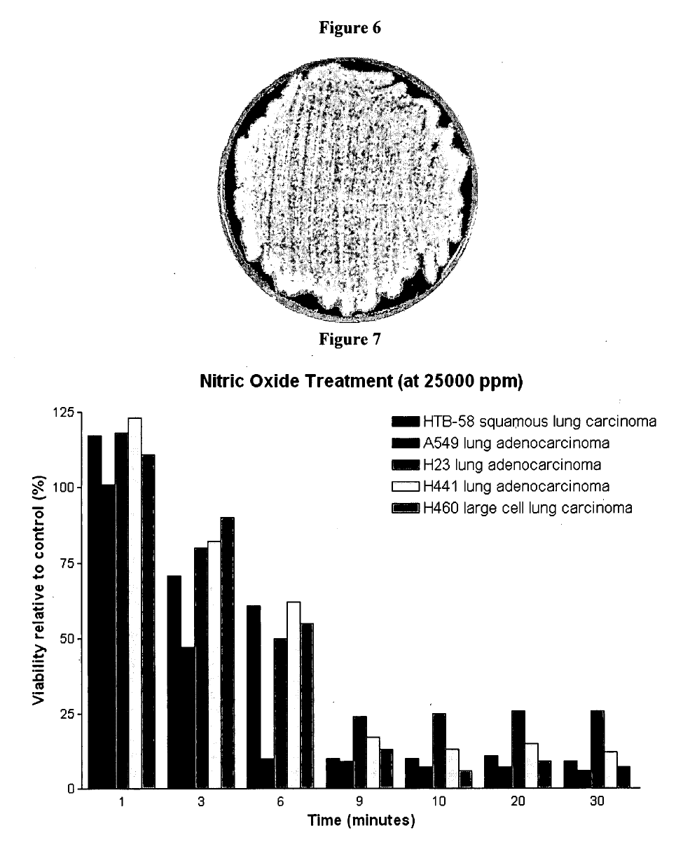

Results show that 25,000 ppm gNO has a profound inhibitory effect on the growth of S. aureus after 1 minute and a possible cidal effect at 3 minutes. FIG. 4 illustrates 1 minute of exposure to gNO at 25,000 ppm; the plate still shows signs of the S. aureus. FIG. 5 illustrates 3 minutes of exposure to gNO at 25,000 ppm; the plate shows no signs of the S. aureus. FIG. 6 illustrates a control plate of S. aureus that was exposed to air.

Further studies may be conducted to optimize the time and dosing. A prospective target would be 20,000 ppm gNO or less for the shortest period of time. A reduction in log three colony forming units per milliliter may be a sufficient in vitro target.

### Example 4

The purpose of this example is to illustrate the tumorcidal activity of high-dosage gNO. We examined the cellular sensitivity to 25,000 ppm of gNO on 5 non-small cell lung cancer (NSCLC) cells lines by an MTS cell proliferation assay.

Human lung cancer cell lines A549, NCI-H23, NCI-H460, HTB-58, H2170, and H441 (commercially available from American Type Culture Collection (“ATCC”), Manassas, Va.) were maintained in culture medium recommended by ATCC. All media were supplemented with 1% penicillin/streptomycin and 10% fetal bovine serum (commercially available from Invitrogen Gibco-BRL, Burlington, Ontario, Canada). All cell lines were incubated in a humidified incubator at 37° C. supplied with 5% carbon dioxide. The cell lines were tested regularly for the absence of Mycoplasma infections. The cells were routinely maintained in 25 cm tissue culture flasks (commercially available from BD Biosciences Discovery Labware, Oakville, Ontario, Canada) and were harvested by 0.25% trypsin (commercially available from Invitrogen-Gibco-BRL) treatment when they were in a logarithmic phase of growth for all experiments.

After harvesting from culture, the A549, NCI-H23, NCI-H460, HTB-58, H2170 and H441 cells were seeded at a density of 5,000 cells per well in pentad in 96-well tissue culture plates (commercially available BD Biosciences Discovery Labware). The cells were incubated for 24 hours in a humidified incubator at 37° C. supplied with 5% carbon dioxide.

After 24 hours, the A549, NCI-H23, NCI-H460, HTB-58, H2170 and H441 cells were treated with 25,000 ppm gNO or air (control) for 1 minute, 3 minutes, 6 minutes, 9 minutes, 10 minutes, 20 minutes, and 30 minutes. Immediately following treatment, the viability of the cells was determined using a colorimetric MTS (3-(4,5-dimethylthiazol-2-yl)-5-(3-carboxymethoxyphenyl)-2-(4-sulfophenyl)-2H-tetrazolium, inner salt) cell proliferation assay (commercially available as the CellTiter 960 Aqueous One Solution Cell Proliferation Assay (MTS) from Promega, Madison, Wis., USA) according to the manufacturer's protocol. Briefly, 20 mL of MTS solution was added to each well and the cells were incubated for 2 hours at 37° C. Absorbance was measured at 490 nm for each well using a micro-plate reader (commercially available from Dynex Technologies, Chantilly, Va., USA). The relative percentage of cell survival (viability) was calculated by dividing the absorbance of treated cells by that of control cells for each experimental time point.

The viability of the A549, NCI-H23, NCI-H460, HTB-58, H2170 and H441 cells after treatment with 25,000 ppm gNO for 1 minute, 3 minutes, 6 minutes, 9 minutes, 10 minutes, 20 minutes, and 30 minutes is shown graphically in FIG. 7. The sensitivity to gNO among the cell lines ranged from IC₅₀ at about 3 minutes for A549, at about 6 minutes for H23, and at about 7 minutes for HTB-58, H460 and H441. Two cell lines, H23 and H441 (at 9 minutes, about 24% and 17% viability) were found to be more resistant to gNO compared to the mean value of the other three NSCLC lines (at 9 minutes, 10.67±2.08% viability).

These results suggest that 25,000 ppm gNO kills between 75-95% of all five cancer cell lines between 6 and 9 minutes. Thus, the shortest time needed to achieve the desired response may be about 6 minutes, about 7 minutes, about 8 minutes, or about 9 minutes.

### Example 5

The purpose of this example is to illustrate the effects on normal human cell lines compared to the effects on cancerous cell lines of high-dosage gNO. The above study was repeated. In addition to the A549, NCI-H23, NCI-H460, HTB-58, H2170 and H441 cells, four other cell lines were added—normal human bronchial epithelial cells (NHBE), normal human endothelial lung vascular cells (NHVE), normal human fibroblast cells (NHLF), and a squamous lung cancer cell line (H2170). The results showed that the normal endothelial cells were rapidly killed in 6 minutes whereas the normal lung fibroblasts still had a 30% viability after 15 minutes of exposure. The data on the cancer cell lines were reproducible and showed that 25,000 ppm gNO kills between 75-95% of all five cancer cell lines between about 6 and about 9 minutes.

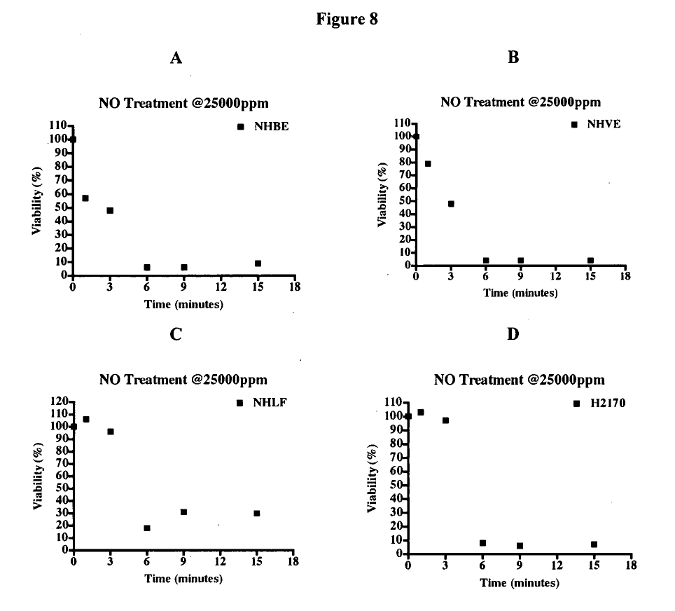

FIG. 8, embodiments A, B, C, and D, illustrate the viability of the three normal human cell lines and one cancerous cell line to which 25,000 ppm gNO is administered for up to 15 minutes. The NHBE cells (embodiment A), experienced a decrease in viability after only 6 minutes of 25,000 ppm gNO administration, resulting in a viability of less than 10%. The NHVE cells (embodiment B) experienced a decrease in viability after only 6 minutes of 25,000 ppm gNO administration, resulting in a viability of less than 10%. The NHLF cells (embodiment C) also experienced a decrease in viability after only 6 minutes of 25,000 ppm gNO administration, resulting in a viability of about 30%. The H2170 cancerous cells experienced a dramatic decrease in viability after only 6 minutes of 25,000 ppm gNO administration, resulting in a viability of less than 10%.

The foregoing detailed description is provided to describe the invention in detail, and is not intended to limit the invention. Those skilled in the art will appreciate that various modifications may be made to the invention without departing significantly from the spirit and scope thereof.

1. A method for preventing cancerous cell phenotypes and growths in an animal comprising the steps of:
providing gaseous nitric oxide; and
administering the gaseous nitric oxide to the animal at one or more administration sites.

2. The method of claim 1, wherein the gaseous nitric oxide is administered in a concentration greater than or equal to about 200 ppm.

3. The method of claim 1, wherein the gaseous nitric oxide is administered for a period of time greater than or equal to about 1 hour.

4. The method of claim 1, wherein the gaseous nitric oxide is administered for a period of time greater than or equal to about 2.5 hours.

5. The method of claim 1, wherein the gaseous nitric oxide is administered in a concentration greater than or equal to about 350 ppm.

6. The method of claim 1, wherein the animal is a mammal.

7. The method of claim 6, wherein the mammal is a human.

8. The method of claim 1, wherein the cancerous cell phenotypes and growths are located in or on the adrenal gland, bladder, bones, brain, breast, cervix, colon, colorectum, esophagus, gastrointestinal tract, heart, kidney, liver, large intestine, lungs, mouth, ovaries, pancreas, parathyroid, pituitary gland, prostate, salivary gland, skin, small intestine, spleen, stomach, thymus, thyroid, testicles, urinary tract, uterus, or vagina.

9. The method of claim 1, wherein the administering step comprises supplying gaseous nitric oxide to the administration site using a device that uses a positive pressure differential to supply the gaseous nitric oxide to the administration site.

10. The method of claim 1, further comprising scavenging gaseous nitric oxide from the one or more administration sites.

11. The method of claim 10, wherein the scavenging step comprises removing the gaseous nitric oxide from the administration site using a negative pressure differential.

12. A method for eradicating cancerous cell phenotypes and growths in an animal comprising the steps of:
providing gaseous nitric oxide; and
administering the gaseous nitric oxide to the animal at one or more administration sites.

13. The method of claim 12, wherein the gaseous nitric oxide is administered in a concentration greater than or equal to about 200 ppm.

14. The method of claim 12, wherein the gaseous nitric oxide is administered for a period of time greater than or equal to about 1 hour.

15. The method of claim 12, wherein the gaseous nitric oxide is administered for a period of time greater than or equal to about 2.5 hours.

16. The method of claim 12, wherein the gaseous nitric oxide is administered in a concentration greater than or equal to about 350 ppm.

17. The method of claim 12, wherein the animal is a mammal.

18. The method of claim 12, wherein the mammal is a human.

19. The method of claim 12, wherein the administration sites are located in or on the adrenal gland, bladder, bones, brain, breast, cervix, colon, colorectum, esophagus, gastrointestinal tract, heart, kidney, liver, large intestine, lungs, mouth, ovaries, pancreas, parathyroid, pituitary gland, prostate, salivary gland, skin, small intestine, spleen, stomach, thymus, thyroid, testicles, urinary tract, uterus, or vagina.

20. The method of claim 12, wherein the administering step comprises supplying gaseous nitric oxide to the administration site using a device that provides a positive pressure differential to supply the gaseous nitric oxide to the administration site.

21. The method of claim 12, wherein between about 75% and about 95% of the cancerous cell phenotypes or growth are killed by the gaseous nitric oxide.

22. The method of claim 12, further comprising scavenging gaseous nitric oxide from the one or more administration sites.

23. The method of claim 22, wherein the scavenging step comprises removing the gaseous nitric oxide from the administration site using a negative pressure differential.

24. A method for eradicating cancerous cell phenotypes and growths in an animal comprising the steps of:
identifying cancerous cell phenotypes or growths in the mammal with a device; and
administering the gaseous nitric oxide to the identified cancerous cell phenotypes or growth with the device, wherein the gaseous nitric oxide is administered at a concentration greater than or equal to about 200 ppm.

25. The method of claim 24, wherein the device is an endoscope
